(19) United States
(12) Patent Application Publication
(10) Pub. No.: US 2006/0135861 A1
(43) Pub. Date: Jun. 22, 2006

(54) Apparatus and method for blood analysis

(75) Inventors: Gerhardus Wilhelmus Lucassen, Eindhoven (NL); Gerwin Jan Puppels, Rotterdam (NL); Marjolein Van Der Voort, Eindhoven (NL); Rolf Wolthuis, Rotterdam (NL)

Correspondence Address: PHILIPS INTELLECTUAL PROPERTY & STANDARDS, 595 MINER ROAD, CLEVELAND, OH, 44143, US

(73) Assignee: Koninklijke Philips Electronics N.V., Eindhoven (NL)

(21) Appl. No.: 10/544,293

(22) PCT Filed: Jan. 19, 2004
(86) PCT No.: PCT/IB04/50034

§ 371 Date: Aug. 3, 2005

(30) Foreign Application Priority Data
Feb. 6, 2003 (EP) 03100249.6

(51) Int. Cl. A61B 5/00

(52) U.S. Cl. 600/315

(57) ABSTRACT

The present invention relates to an analysis apparatus, in particular a spectroscopic analysis apparatus, for blood analysis on vessels. An excitation system (exs) emits an excitation beam to excite a target region. A detection system (dsy) is provided for detecting and analyzing scattered radiation from the target region. Those areas are selected or predetermined so that only scattered radiation from blood in capillaries having a diameter below a predetermined diameter value and/or including an amount of red blood cells below a predetermined cell amount is analyzed. Thus, in contrast to analysis on whole blood or large amounts of blood cells less reabsorption and scattering of Raman light due to red blood cells is obtained. Further, the possibility to directly measure in the blood plasma without interference of the red blood cells, thereby yielding a higher signal-to-noise ratio, is given.

The present invention relates to an analysis apparatus, in particular a spectroscopic analysis apparatus, for blood analysis and a corresponding analysis method.

In general, analysis apparatuses, such as spectroscopic analysis apparatuses, are used to investigate the composition of an object to be examined. In particular, analysis apparatuses employ an analysis, such as a spectroscopic decomposition, based on interaction of the matter of the object with incident electromagnetic radiation, such as visible light, infrared or ultraviolet radiation.

A spectroscopic analysis apparatus comprising an excitation system and a monitoring system is known from WO 02/057759 which is incorporated herein by reference. The excitation system emits an excitation beam to excite a target region during an excitation period. The monitoring system emits a monitoring beam to image the target region during a monitoring period. The excitation period and the monitoring period substantially overlap. Hence the target region is imaged together with the excitation, and an image is formed displaying both the target region and the excitation area. On the basis of this image, the excitation beam can be very accurately aimed at the target region.

WO 96/29925 discloses an apparatus and method of measuring selected analytes in blood and tissue using Raman spectroscopy to aid in diagnosis. More particularly, Raman spectra are collected and analyzed to measure the concentration of dissolved gases and other analytes of interest in blood. Measures include in vivo transdermal and continuous monitoring as well as in vitro blood analysis. Furthermore, a compound parabolic concentrator to increase the amount of detected Raman signal is disclosed.

The problem encountered with the analysis of whole blood Raman spectra is that the signal is almost completely dependent on the amount of hemoglobin. The signal contribution of other analytes is limited to a few percent or less and is therefore measured against the very large background signal, which moreover strongly varies with oxygenation of hemoglobin. Moreover, usually the analyte concentration values in plasma are the parameter of interest, but Raman spectroscopy does not discriminate between intracellularly and extracellularly localized analytes. Under normal physiological circumstances about 35-50% of the blood volume is taken up by red blood cells. Furthermore, when measuring Raman spectra of bulk samples signal collection efficiency will be affected by multiple light scattering by the red blood cells, resulting in a less well defined measuring volume and by absorption of excitation and Raman scattered light.

It is thus an object of the present invention to provide an analysis apparatus and a corresponding analysis method which supply an analysis of a target comprised in the object to be examined more reliably, in particular avoiding the above described problems, having a better signal-to-background ratio and providing signals having a higher signal contribution of other analytes apart from hemoglobin than provided by the known analysis apparatus.

This object is achieved according to the present invention by an analysis apparatus as claimed in claim 1 comprising:
an excitation system for emitting an excitation beam to excite a target region,
a detection system for detecting scattered radiation from the target region generated by the excitation beam and for analyzing the scattered radiation,
   wherein only scattered radiation from blood in capillaries having a diameter below a predetermined diameter value and/or including an amount of red blood cells below a predetermined cell amount is analyzed.

The object is further solved by a corresponding analysis method as claimed in claim 18.

The present invention is based on the idea that spectroscopic analysis on small blood vessels such as capillaries in the skin just below the epidermal junction and/or on vessels having a low amount of red blood cells have specific advantages over analysis on whole blood, in large blood vessels or large amounts of blood cells. One analysis option is that only scattered radiation from selected vessel areas where are only small capillary vessels or vessels having a low amount of red bloods cells are present is detected and analyzed. Another analysis option, which can be employed additionally or alternatively, is to excite only those selected vessel areas or other predetermined areas where only small capillary vessels or vessels having a low red blood cell amount are present, such as in the upper dermis.

Since it is known that in capillary vessels the haematocrit is markedly lower than in larger blood vessels the above mentioned problems are ameliorated by the invention. The ratio of plasma versus red blood cell amount is improved, multiple scattering effects are not appearing since blood cells pass one by one in small capillary vessels and no self-absorption appears since no plasma signal is obtained when a red blood cell passes. Further, an increased signal-to-background ratio can be achieved since due to less red blood cells relatively more plasma is present which increases the ratio.

Further advantages are that the present invention can be advantageously employed to examine in vivo as well as in vitro the composition of blood in capillaries. The analysis can be done directly on the plasma, without interference from red blood cells, thus enhancing the signal-to-noise ratio. Basically, this enables the possibility to detect signal at periods when the detection volume is occupied by plasma and during periods in which red blood cells are in the detection volume the detection or excitation can be stopped or blocked. Moreover, the analysis on plasma better compares to in vitro analysis on blood which is also done on the plasma without the red blood cells.

Light scattering from blood cells itself in small blood vessels is limited which is a problem in whole blood analysis. Still further, reabsorption from the induced Raman light is limited due to the small size while reabsorption is a problem in whole blood analysis. According to the invention the analysis need not to be corrected for different haematocrits which makes the analysis faster and easier. Since, for instance, the absorption length at 920 nm is about 700 μm for an absorption coefficient of 1.46 mm⁻¹. This means that at a 10-15 μm diameter capillary reabsorption is negligible when measuring in capillaries.

Another advantage is that the analysis need not to be corrected for different oxygenations of hemoglobins in the red blood cells which makes the analysis faster and easier as well. The oxygen dissolved in the plasma is only a small fraction (˜4% of the total oxygen in blood).

According to the invention the confocal volume of the excitation beam can be easily fitted to the size of the small blood capillaries using high numeric aperture objective lenses and wavelengths in the near-infrared (NIR) range.

Preferred embodiments of the invention are defined in the dependent claims. A preferred embodiment for in vivo analysis is defined in claim 2, which further comprises:
a monitoring system for emitting a monitoring beam to image the target region,
an image processing unit for processing the image of the target region and for selecting vessel areas in the image showing capillary vessels or vessel portions having a diameter below a predetermined diameter value and/or including an amount of red blood cells below a predetermined cell amount, and
a control unit for controlling the detection system to analyze only scattered radiation from the selected vessel areas and/or for controlling the excitation system to excite only the selected vessel areas or predetermined areas.

Preferred embodiments of the image processing unit are defined in claims 6 to 8. For selecting only vessel areas in the image showing small vessels optical vessel tracking means are provided.

For the selection of vessels or vessel portions having a low red blood cell amount the contrast in the image can be used, for instance from an OPSI (orthogonal polarized spectral imaging) image. When blood is present the use of light that is absorbed by blood gives a dark contrast with respect to the light parts in the image which represents the skin surrounding the blood vessel. If there are no red blood cells, there is no contrast. When there are red blood cells present, these cells can be visualized since there is contrast. It is preferred to acquire the images within a short time to be able to see individual blood cells. However, it is also possible to integrate the acquired data over a certain time and to generate images from integrated data.

Preferably, means for enrichment of plasma signal contribution and/or selection means for a selective analysis of the plasma component are provided, e.g. for analyzing only in the plasma.

According to another embodiment means for stopping or slowing down the blood flow, in particular by pressure squeezing, for instance an inflatable cushion, is provided to control external pressure on the blood vessels. This enables to control the amount of blood cells in the capillaries and to provide for vessels with partly no blood cells present and partly cells present.

A preferred embodiment of the control unit is defined in claim 9. The excitation system is thus controlled to excite only predetermined areas. For instance, in the upper dermis the penetration depth of the imaging technique is less than 300 μm.

An embodiment of the analysis apparatus for in vitro analysis is defined in claim 11 which further comprises a sample holding system comprising a capillary carrier containing the blood to be analyzed. Preferred embodiments thereof are defined in claims 12 and 13. This in vitro analysis apparatus needs a little amount of blood, reduces scattering problems in whole blood, reduces reabsorption problems and has a high throughput.

Preferably, capillaries are used according to the present invention having a diameter value of less than 15 μm, in particular less than 10 μm. Typical diameters for small vessels are in the range from 5 to 10 μm. The normal size of red blood cells is 7 μm in diameter and 2-3 μm in thickness.

Further, according to a preferred embodiment blood is analyzed with a red blood cell amount having a haematocrit value below 0.4. The haematocrit value is defined as the volume occupied by red blood cells to the total blood volume. Since the haematocrit in capillaries is markedly lower, in particular lower than 0.35, than in larger blood vessels which have a haematocrit in the range of 0.35-0.5, it is an appropriate criterion for selection of vessel areas. It shall be noted that the amount of red blood cells for haematocrit 0.35 is about 3.5·10¹² red blood cells per liter.

There are several ways to trigger Raman signal analysis. According to one embodiment in the region of interest including at least part of a small (capillary) blood vessel the velocity and direction of flowing cells can be analyzed. From the velocity of the cells and the distance a trigger can be provided to the Raman detection system to collect a signal when the cells are absent in the Raman measuring point, which can, for instance, be in the middle of the length of the blood vessel, and not to collect a signal when red blood cells are present there. The detection system can thus be controlled efficiently.

By measuring in vivo and in plasma by using control unit triggering further advantages can be achieved. For example, a problem is found in the determination of cholesterol from whole blood. Since 40% of the cholesterol remains in the cell membrane, a different concentration results when measuring in whole blood or in plasma. Further, the measurements can be directly compared to in vitro reference measurements which can not be done on whole blood unless the cells flow by one by one.

According to another embodiment the intensity of the (elastically) scattered radiation is exploited. This intensity is high when there is a cell in the measurement position and lower if cells are not present in the Raman measurement position. Thus, again, the detection system can be controlled efficiently by use of said intensity information.

It is further advantageous that the excitation period during which the target region is excited and the monitoring period during which the target region is imaged by the monitoring beam substantially overlap as described in WO 02/057759, particularly in the embodiment using intensity information retrieved from scattered radiation for control of the detection system.

The analysis apparatus according to the present invention can be a two-laser or a one-laser apparatus. In the two-laser apparatus one laser is used to produce the excitation beam while a different laser is used to emit the monitoring beam. In the one-laser embodiment the original output beam generated by a radiation source, i.e. a laser, is preferably split into the monitoring beam and the excitation beam by appropriate optical separation means. Further, an OPSI (orthogonal polarized spectral imaging) arrangement, preferably comprising one or two light sources (e.g. 2 LEDs of different color, or 1 white light source) can be employed in the monitoring system as described in WO 02/057759.

Other suitable options for the monitoring systems are for example an optical coherence tomography (OCT) arrangement, an optical Doppler tomography (ODT) arrangement, a photo-acoustic imaging (PAI) arrangement, or a multiphoton microscopy (MPM) arrangement. Notably, the OCT, ODT and PAI arrangements give good results for monitoring blood vessels or other target areas that lie deeper, up to several millimeters, under the skin surface. The MPM arrangement in conjunction with confocal imaging provides a high resolution where details of 3-5 μm are rendered well visible. The MPM arrangement is further suitable for imaging details at a depth up to 0.25 mm.

The invention will now be explained in more detail with reference to the drawings in which

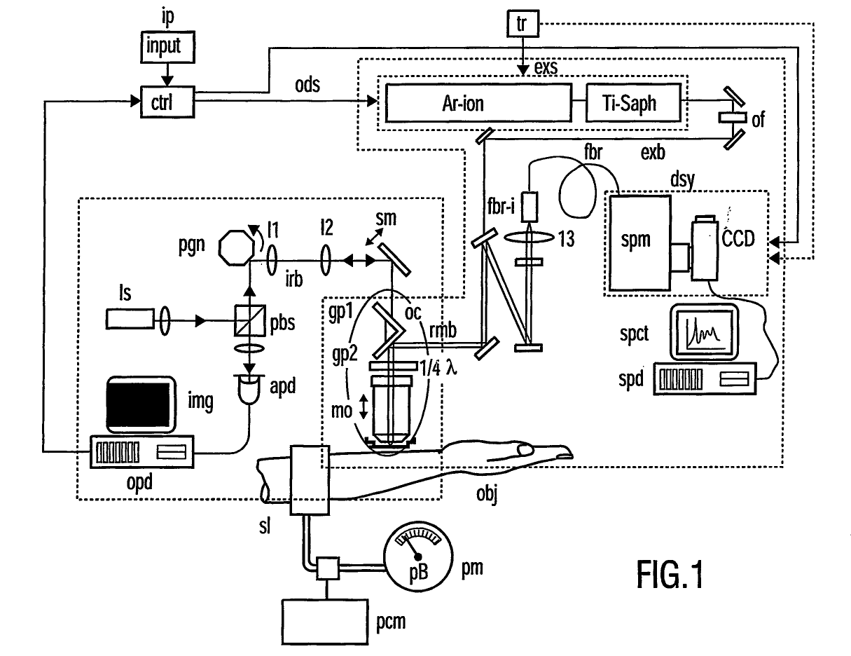

FIG. 1 shows a graphic representation of an in vivo analysis apparatus according to the present invention,

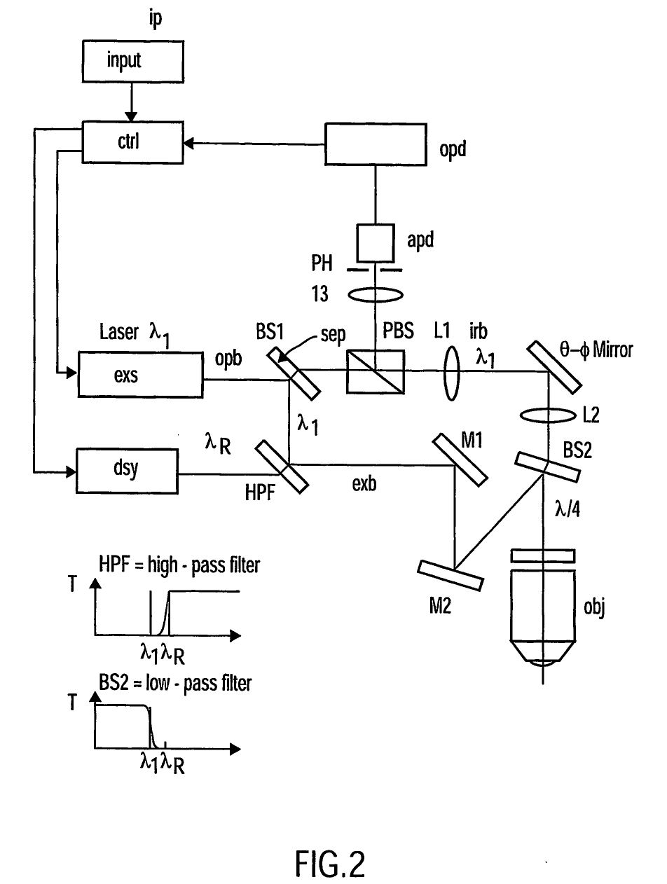

FIG. 2 shows a graphic representation of another embodiment of an in vivo analysis system according to the present invention,

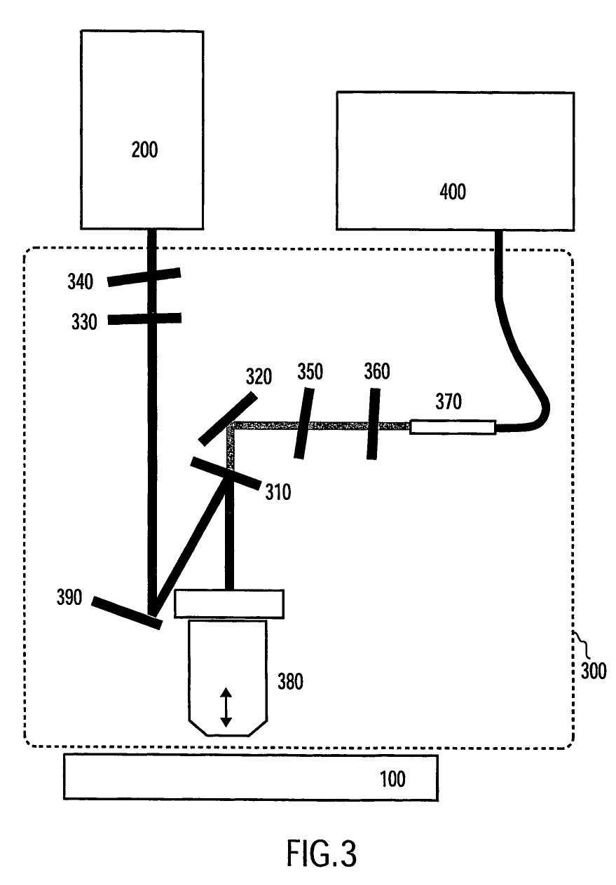

FIG. 3 shows a graphic representation of an in vitro analysis apparatus according to the present invention,

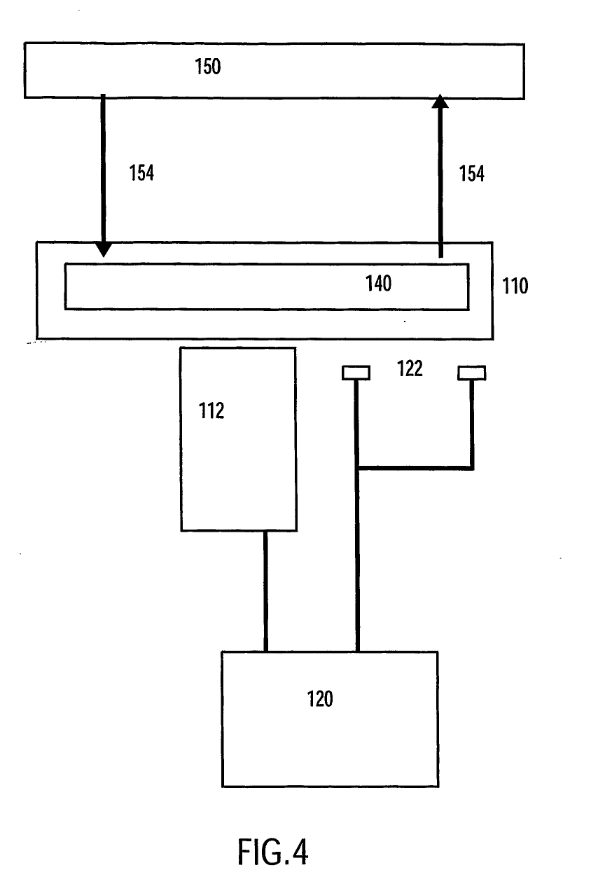

FIG. 4 shows graphic representation of a sample holding device of the embodiment shown in FIG. 3,

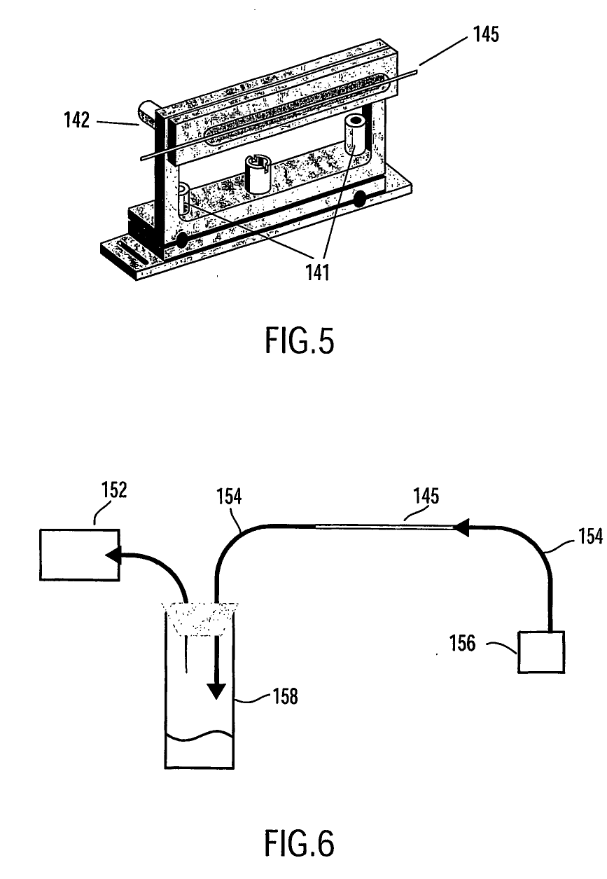

FIG. 5 shows an example of a capillary holder of the embodiment shown in FIG. 3,

FIG. 6 shows a graphic representation of a capillary holder of the embodiment shown FIG. 3, and

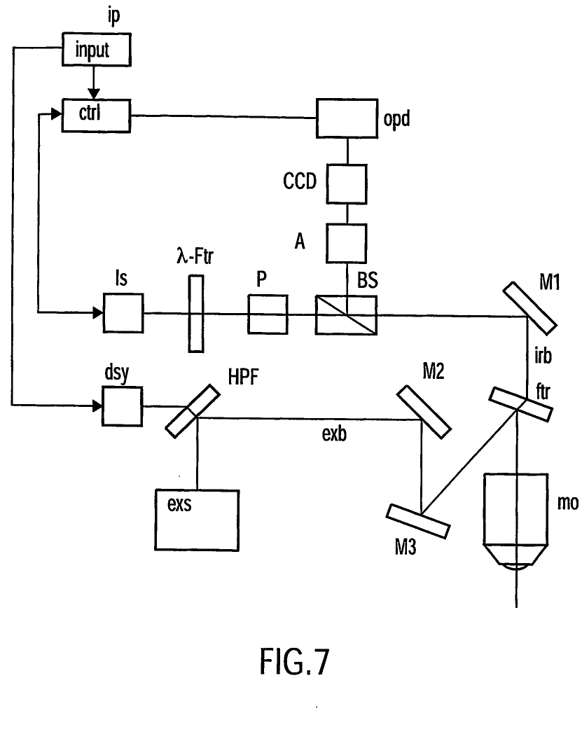

FIG. 7 shows a graphic representation of an OPSI arrangement for in vivo analysis according to the present invention.

FIG. 1 is a graphic representation of an analysis system in accordance with the invention. The analysis system includes the monitoring system incorporating a light source (ls) with optical imaging system (lso) for forming an optical image of the object (obj) to be examined. The optical imaging system (lso) forms a confocal video microscope. In the present example the object is a piece of skin of the forearm of the patient to be examined. The analysis system also includes a multi-photon, non-linear or elastic or inelastic scattering optical detection system (ods) for spectroscopic analysis of light generated in the object (obj) by a multi-photon or non-linear optical process. The example shown in FIG. 1 utilizes in particular an inelastic Raman scattering detection system (dsy) in the form of a Raman spectroscopy device. The term optical encompasses not only visible light, but also ultraviolet radiation and infrared, especially near-infrared radiation.

The light source (ls) is formed by an 834 nm AlGaAs semiconductor laser whose output power on the object to be examined, that is, the skin, amounts to 15 mW. The infrared monitoring beam (irb) of the 834 nm semiconductor laser is focussed in the focal plane in or on the object (obj) by the optical imaging system in the exit focus. The optical imaging system includes a polarizing beam splitter (pbs), a rotating reflecting polygon (pgn), lenses (11, 12), a scanning mirror (sm) and a microscope objective (mo). The focussed monitoring beam (irb) is moved across the focal plane by rotating the polygon (pgn) and shifting the scanning mirror (sm). The exit facet of the semiconductor laser (ls) lies in the entrance focus. The semiconductor laser (ls) is also capable of illuminating an entrance pinhole in the entrance focus. The optical imaging system conducts the light that is reflected from the focal plane as a return beam, via the polarizing beam splitter (pbs), to an avalanche photodiode (apd). Furthermore, the microscope objective (mo) is preceded by a λ/4-plate so that the polarization of the return beam is perpendicular to the polarization of the monitoring beam. The polarizing beam splitter (pbs) thus separates the return beam from the monitoring beam.

An optical display unit (opd) utilizes the output signal of the avalanche photodiode (apd) to form the image (img) of the focal plane in or on the object to be examined, said image being displayed on a monitor. In practice the optical display unit is a workstation and the image is realized by deriving an electronic video signal from the output signal of the avalanche photodiode (apd) by means of the processor of the workstation. This image is used to monitor the spectroscopic examination, notably to excite the target region such that the excitation area falls onto the target region and receiving scattered radiation from the target region.

The Raman spectroscopy device includes an excitation system (exs) which is in this case constructed as an Ar-ion/Ti-sapphire laser which produces the excitation beam in the form of an 850 nm (or 785 nm or 810 nm) infrared beam (exb). The Ti-sapphire laser is optically pumped with the Ar-ion laser. Light of the Ar-ion laser is suppressed by means of an optical filter (of). A system of mirrors conducts the excitation beam to the optical coupling unit (oc) and the optical coupling unit conducts the excitation beam along the monitoring beam (irb) after which the microscope objective focuses it in the focal plane at the area of the focus of the monitoring beam. The optical coupling unit (oc) forms the beam combination unit. The optical coupling unit conducts the excitation beam along the optical main axis of the microscope objective, that is, along the same optical path as the monitoring beam.

The Raman scattered light is reflected to the entrance of a fiber (fbr) by the optical coupling unit (oc). The Raman scattered infrared light is focussed on the fiber entrance in the detection pinhole by the microscope objective (mo) and a lens (l3) in front of the fiber entrance (fbr-i). The fiber entrance itself acts as a detection pinhole. The optical imaging system establishes the confocal relationship between the entrance focus, where the semiconductor laser (ls) is present, the exit focus at the area of the detail of the object (obj) to be examined and the detection focus in the fiber entrance (fbr-i). The fiber (fbr) is connected to the input of a spectrometer (spm) with a CCD detector (CCD). The spectrometer with the CCD detector is incorporated into the detector system (dsy) which records the Raman spectrum for wavelengths that are smaller than approximately 1050 nm. The output signal of the spectrometer with the CCD detector represents the Raman spectrum of the Raman scattered infrared light. In practice this Raman spectrum occurs in the wavelength range beyond 860 nm. The signal output of the CCD detector is connected to a spectrum display unit (spd), for example a workstation which displays the recorded Raman spectrum (spct) on a monitor

In practice the functions of the optical display unit and the spectrum display unit can be carried out by means of the same workstation. For example, separate parts (windows) of the display screen of the monitor are used for simultaneous display of the optical image and the Raman spectrum. Regarding further details of the analysis apparatus in general and the function thereof reference is made to the above mentioned WO 02/057759.

According to the invention a control unit (ctrl) is provided which controls either the detection system (dsy) and/or the excitation system (exs) such that only scattered radiation from selected vessel areas is analyzed and/or that only selected vessel areas or predetermined areas are excited. The control unit (ctrl) is preferably triggered by the image processing unit (opd) where the selected vessel areas are selected in the image (img). The selected vessel areas are selected such that they inhibit only vessels or vessel portions having a diameter below a predetermined diameter value, such as having a diameter value lower than 15 μm, or even lower than 10 μm. Another criterion for selecting vessel areas is the amount of red blood cells which should be below a predetermined cell amount, such as below haematocrit value 0.35 since larger vessels have also a larger haematocrit value above 0.35. Those selection criteria can be set by an input unit (ip), for instance can be stored in a memory or inputted by a user.

By measuring in vivo and in plasma by using the control unit triggering further advantages can be achieved: For example, a problem is found in the determination of cholesterol from whole blood. Since 40% of the cholesterol remains in the cell membrane, a different concentration results when measuring in whole blood or in plasma. Thus cholesterol determination in whole blood is a problem due to the fact that 40% remains in the cells and the measurements can be directly compared to in vitro reference measurements, which cannot be done on whole blood unless the cells flow by one by one.

Preferably, the in vivo blood analysis is further enabled to enrich plasma signal contribution and/or for selective analysis of the plasma component (i.e. only in plasma). Further, a time-resolved excitation or detection can be foreseen by a trigger unit (tr). Still further, means for stopping or slowing down the blood flow can be provided, e.g. by pressure squeezing, so as to allow the selection of cell free spots to measure.

Further, in addition or alternatively, the areas to be excited by the excitation system (exs) can also be predetermined, for instance by input at the input unit (ip) or stored therein. Capillaries can generally be found on various locations all over the skin of a person's body where the capillaries have different size, shape and depth position in the skin. Good candidate locations are: under the tongue, on the inner lip in the mouth, the inner side of the cheek, on the nose, on the earlobe, near the temple, under the eye, on the inner side of the upper arm, on the volar aspect of the forearm, on the food under the ankle, on the hand, on the finger nail bed, on the finger tip or on the back of the hand. One or more of these areas can be predetermined so that the control unit (ctrl) controls the excitation system such that only the predetermined area is excited.

A local analysis of a composition, in particular a non-invasive blood analysis, can be employed by the invention, but also an in vitro or ex vivo blood analysis through a small capillary is possible.

For stopping or slowing down the blood flow an inflatable sleeve (sl) for pressure squeezing the forearm of the patient is provided, connected to a pressure meter (pm) and a pressure control unit (pcu). This enables to control the amount of blood cells in the capillaries and to provide for vessels with partly no blood cells present and partly cells present.

While FIG. 1 shows an embodiment of an analysis apparatus having two lasers, FIG. 2 diagrammatically shows an embodiment of the analysis apparatus according to the invention including an optical separation system. A laser at λ₁ forms the radiation source that is used for confocal imaging and simultaneously for Raman excitation. The beam is split in two by the optical separation system (sep) formed by an (e.g. 20-80%) beam splitter (BS1). Part is used for confocal imaging, the other part is used for Raman excitation. The monitoring beam is linearly polarized by the polarizing beam splitter (PBS). The scanning beam path in the confocal video microscope is deflected in x-y plane by the Θ-Φ mirror to form the image. Lenses L1 and L2 are used for beam expansion and L2 is used to image the central part of the Θ-Φ mirror on to the entrance pupil of the microscope objective (mo). In this way laser light reflected of the Θ-Φ mirror always enters the objective at the same position, irrespective of the actual Θ-Φ position of the Θ-Φ mirror.

The linearly polarized monitoring (λ₁) beam is transformed to circularly polarized light by the quarter wave plate λ/4. The Raman excitation beam is reflected at the high pass filter (HPF) and directed towards the objective via the mirrors (M1, M2) and reflecting beamsplitter (BS2). On the return path reflected light from the object is transformed to linearly polarized light again however, shifted by 90° orientation, with respect to the polarization orientation of the incoming beam. The transmitted light (partly the monitoring beam and partly the elastically scattered Raman light) trough the reflecting beam splitter (BS2) is then deflected by the polarizing beam splitter (PBS) towards the APD detector to form the image and the Raman spot in the image. Elastically and inelastically scattered Raman light from the object is reflected at the BS2. The inelastically scattered Raman light (λ_{R}) is transmitted through the high pass filter HPF and directed towards the Raman detection path. The beamsplitter (BS2) can be exchanged by a spot reflector.

As described above regarding the first embodiment shown in FIG. 1 a control unit (ctrl) and an input unit (ip) are provide for control of the detection system (dsy) and/or the excitation system (exs) based on information received from the imaging system (opd) and/or the input unit (ip) in the way described above.

In the following an in vitro analysis apparatus according to the invention shall be described which is shown in FIGS. 3 to 6. This apparatus is designed to measure Raman spectra in small volumes of fluids and suspensions, hereinafter called sample, in particular blood. The device is particularly suitable for samples with a high absorption and/or turbidity. The influence of absorption of incident laser light and self-absorption scattered light, in particular Raman scattered light, is minimized by reducing the volume from which Raman signal is collected to a few cubic micrometers at the surface of the sample. The total sample volume, on the other hand, is increased by moving the sample through the volume from which the Raman signal is collected. Self-absorption is thus minimized due to the short optical pathlength of the scattered light while the sample volume is increased by scanning the sample. Energy is deposited into a large sample volume, thereby minimizing potential heating of the sample by absorption of incident laser light, which could give rise to unwanted changes in the light scattering characteristics of the sample. Also the large sample volume ensures that a representative Raman signal is obtained of inhomogeneous samples, such as e.g. blood.

The apparatus is shown as a block diagram in FIG. 3. It comprises a sample handling device (100), a Raman excitation source (200), a spectral analyzer (400) and optics (300) for shaping the laser-beam and/or adjusting its spectral characteristics and/or adjusting its polarization parameters and Raman scattered light.

The output from the Raman excitation source (200), preferably a laser and for blood analysis preferably a laser emitting light at a wavelength >600 nm (typically a Ti:Sapph-laser (Coherent), pumped by an Argon-ion laser (Coherent) is used, emitting continuous laser light at a wavelength of 785 nm) is filtered with a dielectric band pass filter (340) which only transmits light in a narrow wavelength region around the wavelength of the laser light and which efficiently blocks wavelengths greater than the laser wavelength, preferably wavelengths that are greater by more than 5 nm than the laser wavelength. The linearly polarized laser beam from the laser (200) is changed into a circular polarized beam using a waveplate (330). Using circular polarized light instead of linearly polarized light has the advantage that effects on the measured signal of the usually polarization dependent signal detection efficiency of a Raman setup are minimized.

The laser beam from the laser (200) is directed to a microscope objective (380) by a mirror (390) and a dielectric filter (310) which efficiently reflects the laser light, but which efficiently transmits light at wavelengths greater than the laser wavelength, preferably starting at wavelengths that are 5 nm greater than the laser wavelength. The microscope objective (380) focuses the laser light into a capillary of the sample holding device (100) containing the sample to be studied. By translating the microscope objective (380) along its optical axis the position of the focus within the capillary can be changed.

The back-scattered light is collected by the microscope objective (380) and collimated. The collimated light falls on the low pass filter (310). The back-scattered laser light and Rayleigh scattered light are mainly reflected, the red shifted Raman scattered light is transmitted. The Raman light is steered with a high reflective mirror (320) towards a holographic notch filter (350), preferably having an optical density of about 6, for further suppression of the laser and Rayleigh scattered light. The Raman light passing the notch filter (350) is focused with a lens (360) on the core of an optical fiber (370). This fiber guides the Raman light into a spectral analyzer (400), in particular a multichannel optical spectrometer, for spectral analysis. The core of the optical fiber is used as a means to limit the measurement volume. The same can be achieved by focusing the Raman scattered light onto a small aperture which forms the entrance to the spectrometer.

An embodiment of the sample holding device (100) is shown in FIG. 4, an embodiment of a capillary holder (140) used therein is shown in FIG. 5. The capillary (145) is placed in a capillary holder (140) equipped with setscrews (141, 142) for vertical alignment (141), perpendicular to the optical plane, and for horizontal alignment (142), such that when the capillary is moved horizontally and perpendicular to the optical axis of the microscope objective, the laser focus remains inside the capillary. The capillary (145) can be either exchangeable or permanently mounted.

The capillary holder (140) is placed on a translation stage (110) which is driven with a piezo-friction motor (112). This stage (110) moves back and forth between two end points set by end switches (122). The signal from the end switches (122) is translated into the motor direction by control electronics (120). The motor speed is also set by the control electronics (120).

The capillary (145) is positioned such that the optical axis of the microscope objective (380) is perpendicular to the side of the capillary (145). The capillary (145) is mounted in the capillary holder (140) allowing optimal positioning such that the focus of the microscope objective (380) falls within the capillary (145) and enabling continuous translation back and forth along the long axis of the capillary (145) while maintaining the focus of the microscope objective (380) within the capillary (145).

For the particular purpose of measuring small blood samples (<200 μl; currently limited by the tube diameter, the capillary volume=12.5 μl; a diameter of 50 μm capillary leads to a capillary volume of 12.5 nl) the capillary (145) is equipped with sample supply means (150) comprising tubes (154) on both sides as shown in FIG. 6. One side is connected to a sample injection port (156), compatible with luer type syringes, the other side is connected with a waste container (158) and a vacuum pump (152). The vacuum pump (152) delivers suction at the injection port (156) allowing easy injection of the sample into the capillary (145) for measurement.

The embodiment as described enables in vitro measurement of highly absorbing fluids/suspensions and strongly scattering fluids/suspensions by limiting the optical pathway of scattered light inside the sample. It thereby reduces the need for difficult signal corrections to be applied to correct for often wavelength dependent self-absorption or scattering in the sample, which may differ from one sample to the next, e.g. because of differences in hematocrit and/or the oxygen saturation of blood.

FIG. 14 diagrammatically shows a further embodiment of the analysis apparatus according to the invention wherein the monitoring system is an orthogonal polarized spectral imaging arrangement. This embodiment combines imaging by OPSI and Raman spectroscopy. For orthogonal polarized spectral imaging (OPSI) a light source is used at a specific wavelength band. To achieve this a white light source is filtered by a band pass filter (λ-Ftr). The light is linearly polarized by the polarizer (P). The light is then focused in the object by the objective lens (Obj). The reflected light is detected through an analyzer at orthogonal polarization orientation. This means that only depolarized light is detected which originates from multiply (diffusely) scattered light deep in the turbid object (tissue). The back scattering of these photons produces a sort of ‘backlight illumination’ which gives a more or less homogenous brightness in the image at the CCD detector (CCD see FIG. 1). By proper selection of the wavelength (λ-Ftr) corresponding to (partly) absorption in shallow objects (such as capillaries in skin) these objects in contrast appear dark (through absorption) on a bright background. A Raman excitation beam can be coupled in the OPSI image in a similar fashion as in confocal imaging using a filter or other beam combination unit. The advantage of OPSI is especially its compactness and low cost. As described with reference to the other embodiments a control unit for control of the excitation system (ls) and/or the detection system (dsy) are provided.

1. An analysis apparatus, in particular a spectroscopic analysis apparatus, for blood analysis comprising:
an excitation system for emitting an excitation beam to excite a target region, and
a detection system for detecting scattered radiation from the target region generated by the excitation beam and for analyzing the scattered radiation,
wherein only scattered radiation from blood in capillaries having a diameter below a predetermined diameter value and/or including an amount of red blood cells below a predetermined cell amount is analyzed.

2. An analysis apparatus as claimed in claim 1, further comprising:
a monitoring system for emitting a monitoring beam to image the target region,
an image processing unit for processing the image of the target region and for selecting vessel areas in the image showing capillary vessels or vessel portions having a diameter below a predetermined diameter value and/or including an amount of red blood cells below a predetermined cell amount, and
a control unit for controlling the detection system to analyze only scattered radiation from the selected vessel areas and/or for controlling the excitation system to excite only the selected vessel areas or predetermined areas.

3. An analysis apparatus as claimed in claim 2, further comprising means for enrichment of plasma signal contribution.

4. An analysis apparatus as claimed in claim 2, further comprising selection means for a selective analysis of the plasma component.

5. An analysis apparatus as claimed in claim 2, further comprising means for stopping or slowing down the blood flow, in particular by pressure squeezing.

6. An analysis apparatus as claimed in claim 2,
wherein the image processing unit is adapted for selecting vessel areas in the image showing capillary vessels or vessel portions having a diameter below a predetermined diameter value by use of optical vessel tracking means.

7. An analysis apparatus as claimed in claim 2, wherein the image processing unit is adapted for selecting vessel areas in the image showing capillary vessels or vessel portions including an amount of red blood cells below a predetermined cell amount by use of the contrast in the image.

8. An analysis apparatus as claimed in claim 2, wherein the image processing unit is adapted for retrieving velocity and distance information of red blood cells in the image and
wherein the control unit is adapted for controlling the detection system by use of said velocity and distance information.

9. An analysis apparatus as claimed in claim 2, wherein the control unit is adapted for controlling the excitation system to excite only predetermined areas in the upper dermis, in particular by use of a penetration depth of less than 300 μm.

10. An analysis apparatus as claimed in claim 2, wherein the detection system is adapted for retrieving intensity information from the scattered radiation and
wherein the control unit is adapted for controlling the detection system by use of said intensity information.

11. An analysis apparatus as claimed in claim 1, further comprising a sample holding system comprising a capillary containing the blood to be analyzed.

12. An analysis apparatus as claimed in claim 11, wherein said capillary is adapted for moving along its longitudinal axis and/or along the direction of the incoming excitation beam.

13. An analysis apparatus as claimed in claim 11, further comprising means for causing a flow of blood through the capillary.

14. An analysis apparatus as claimed in claim 1, wherein said predetermined diameter value is 15 μm, in particular 10 μm.

15. An analysis apparatus as claimed in claim 1, wherein said predetermined blood cell amount is below haematocrit 0.35.

16. An analysis apparatus as claimed in claim 1, further comprising a radiation source to emit an output beam and an optical separation system to separate the monitoring beam and the excitation beam from the output beam.

17. An analysis apparatus as claimed in claim 1, further comprising trigger means for triggering of the excitation system and/or the detection system for time-resolved excitation of the target region and/or for time-resolved detection of scattered radiation from the target region.

18. An analysis method, in particular a spectroscopic analysis method, for blood analysis on vessels comprising the steps of:
emitting an excitation beam to excite a target region,
detecting scattered radiation from the target region generated by the excitation beam,
analyzing the scattered radiation,
wherein only scattered radiation from blood in capillaries having a diameter below a predetermined diameter value and/or including an amount of red blood cells below a predetermined cell amount is analyzed.
